(19) United States
(12) Patent Application Publication
(10) Pub. No.: US 2010/0092411 A1
(43) Pub. Date: Apr. 15, 2010

(54) HYDROPHOBIC ZINC OXIDE POWDER

(75) Inventors: Stipan Katusic, Bad Soden (DE); Guido Zimmermann, Bruehl (DE); Juergen Meyer, Stockstadt (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: Evonik Degussa GmbH, Essen (DE)

(21) Appl. No.: 12/445,474

(22) PCT Filed: Aug. 1, 2007
(86) PCT No.: PCT/EP07/57983

§ 371 Date: Apr. 14, 2009

(30) Foreign Application Priority Data
Nov. 2, 2006 (DE) 10 2006 051 635.4

(51) Int. Cl. A61K 8/27

(52) U.S. Cl. 424/59

(57) ABSTRACT

Hydrophobic zinc oxide powder in the form of partially or completely coated aggregates of zinc oxide primary particles having a carbon content of from 0.4 to 1.5% by weight, based on the hydrophobic zinc oxide powder, and a BET surface area of from 25 to 100 m²/g, where the coated aggregates are present to 0 to <10% in a circular form, 15 to <30% in an ellipsoidal form, to <30% in a linear form, 40 to <70% in a branched form, and the sum of the different forms is 15 100% and the coating comprises chemically bonded linear and/or branched monoalkylsilyl groups Si-alkyl and/or dialkylsilyl groups Si-(alkyl)₂ having 1 to 20 carbon atoms.

The invention relates to a hydrophobic zinc oxide powder, to its preparation and to its use.

Hydrophobic zinc oxide powders are known in the prior art. U.S. Pat. No. 5,486,631 discloses hydrophobic zinc oxide particles which are produced by reacting zinc oxide with a hydrophobicizing agent of the general formula R—Si—[—(—O—SiMe₂-)ₐ-OR′]₃, where R is an alkyl radical having 1 to 10 carbon atoms, R′ is methyl or ethyl and a is 4 to 12. In this process, the choice of zinc oxide is not critical. However, in order to satisfy the requirements of cosmetic formulations, such as transparency and UV absorption, the hydrophobicizing agent has to meet the abovementioned requirements with regard to its structure. These requirements restrict the usability in cosmetic formulations.

EP-A-1508599 discloses a hydrophobic zinc oxide powder which can indeed be obtained using a large number of hydrophobicizing agents, but whose BET surface area is restricted to 18±5 m²/g and whose carbon content is restricted to 0.5 to 1.0% by weight. The dispersibility and transparency of such a powder in cosmetic formulations is in need of improvement.

It was an object of the present invention to provide hydrophobic zinc oxide powders whose usability in cosmetic formulations is not restricted, i.e. which bring about no negative interactions with other constituents of the formulation, can be readily incorporated into the formulation and have a high UV absorption and high transparency.

It was furthermore an object of the invention to provide a process for the preparation of the hydrophobic zinc oxide powder.

The invention provides a hydrophobic zinc oxide powder in the form of partially or completely coated aggregates of zinc oxide primary particles with a carbon content of from 0.4 to 1.5% by weight, based on the hydrophobic zinc oxide powder, and a BET surface area of from 25 to 100 m²/g, where
the coated aggregates are present to 0 to <10% in a circular form, 15 to <30% in an ellipsoidal form, 15 to <30% in a linear form, 40 to <70% in a branched form, and the sum of the different forms is 100% and
the coating comprises chemically bonded linear and/or branched alkylsilyl groups having 1 to carbon atoms.

Surprisingly, it has been found that such a powder has excellent dispersibility in cosmetic formulations. It is therefore surprising, because the person skilled in the art could not assume that the form of the aggregates in the zinc oxide powder according to the invention, together with the low carbon content on the surface in a BET surface area range from 25 to 100 m²/g would show such an effect. The effect can currently not be explained exactly, but it appears that a high proportion of branched aggregates is co-decisive.

Preferably, the proportion of the circular form is 0 to 5%, of the ellipsoidal form 20 to 25%, of the linear form 20 to 25% and of the branched form from 45 to 60%.

The aggregates are divided into the abovementioned forms by image analysis of transmission electron micrographs. For this, about 1000 to 2000 aggregates are evaluated using a camera. The definition of the parameters is in accordance with ASTM3849-89.

The aggregates of the zinc oxide powder according to the invention are present in circular, ellipsoidal, linear and branched forms.

Furthermore, it has been found that the hydrophobic zinc oxide powder according to the invention can preferably have aggregates with an average projected aggregate area of 8000-30 000 nm², an equivalent circle diameter (ECD) of 70-300 nm and an average circumference of 500-2000 nm. These values are likewise determined in accordance with ASTM3849-89.

The carbon content of the hydrophobic zinc oxide powder according to the invention is 0.4 to 1.5% by weight. Within this range, the dispersibility in cosmetic formulations is at its best. Preferably, the carbon content is 0.6 to 1.0% by weight.

The carbon content of the hydrophobic zinc oxide powder according to the invention originates from the alkylsilyl groups on the surface of the aggregates. The alkylsilyl groups have preferably 1 to 10 carbon atoms, particularly preferably 4 to 8 carbon atoms. The type of alkyl groups can be determined, for example, by NMR spectroscopy.

The structures A-J are possible alkylsilyl groups according to the invention. Here, the oxygen atom of the —O—Si bond in each case represents an oxygen atom of the zinc oxide surface.

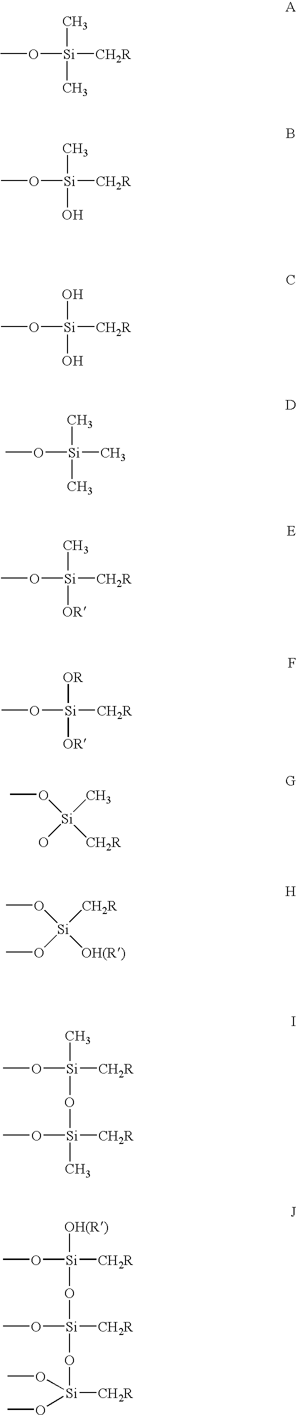

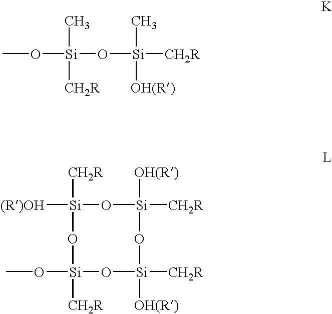

The BET surface area of the hydrophobic zinc oxide powder according to the invention is preferably 25 to 35 m²/g.

Furthermore, the hydrophobic zinc oxide powder preferably has a proportion of lead of at most 20 ppm, of arsenic of at most 3 ppm, of cadmium of at most 15 ppm, of iron of at most 200 ppm, of antimony of at most 1 ppm and of mercury of at most 1 ppm.

The invention further provides a process for the preparation of the hydrophobic zinc oxide powder according to the invention in which a zinc oxide powder in the form of aggregated primary particles with a BET surface area of from 25 to 100 m²/g, in which the aggregates are present to 0-10% in a circular form, to 30-50% in an ellipsoidal form, to 30-50% in a linear form and to 20-30% in a branched form, is sprayed with one or more silanizing agents which contain 1 to 20 carbon atoms, which may optionally be dissolved in an organic solvent, and the mixture is then thermally treated at a temperature of from 120 to 200° C., preferably 140 to 180° C., over a period of from 0.5 to 2 hours, preferably 1 to 1.5 hours.

The process according to the invention can preferably be carried out in a protective-gas atmosphere, for example of nitrogen.

Furthermore, the processes according to the invention can be carried out continuously or discontinuously in heatable mixers and dryers with spray devices, for example in ploughshare mixers, disc dryers, fluidized-bed dryers or moving-bed dryers.

The zinc oxide powder used can be prepared, for example, according to the process described in DE-A-10343728.

This is a fumed zinc oxide powder which is present in the form of aggregates, where the aggregates are constructed from particles of varying morphology, and where the aggregates are present to 0-10% in a circular form, 30-50% in an ellipsoidal form, 30-50% in a linear form and to 20-30% in a branched form.

Morphology is to be understood as meaning both isotropic and anisotropic particles. They can be, for example, spherical or largely spherical particles, bulbous particles, rod-shaped particles or needle-shaped particles. It is essential that the aggregates consist of different particles and these particles are joined together by sintered areas.

Zinc oxide powders can preferably be used which have an approximately identical proportion of 30-40% of ellipsoidal and linear forms and a lower proportion of branched forms of 20-25% and of circular forms of 2-6%.

The zinc oxide powder used can preferably have a tamped density, determined in accordance with DIN ISO 787/11, of at least 150 g/l. Particularly preferably, a tamped density can be between 250 and 350 g/l.

Furthermore, it may be advantageous if the zinc oxide powder used consists of aggregates which have an average projected aggregate area of less than 10 000 nm², an equivalent circle diameter (ECD) of less than 100 nm and an average circumference of less than 600 nm. These sizes can be obtained by image analysis of about 1000 to 2000 aggregates from transmission electron micrographs.

Particularly preferably, a zinc oxide powder may be used whose average projected aggregate area is 2000 to 8000 nm², whose equivalent circle diameter (ECD) is between 25 and 80 nm and whose average circumference is between 200 and 550 nm.

If such a zinc oxide powder is used, then it is surprisingly found that the proportions of the aggregate forms of zinc oxide powder used and the resulting hydrophobic zinc oxide powder are different. Thus, the process according to the invention leads to, in particular, the proportions of the linear and ellipsoidal forms being smaller in the process product than in the material used. The higher proportion of branched aggregates in the process product, in turn, can be used as a criterion for the good dispersibility.

Silanizing agents which can be used are preferably
haloorganosilanes of the type X₃Si (CₙH₂ₙ₊₁), X₂ (R′)Si(CₙH₂ₙ₊₁), X(R′)₂Si(CₙH₂ₙ₊₁), X₃Si (CH₂)ₘ—R′(R)X₂Si(CH₂)ₘ—R′, (R)₂XSi(CH₂)ₘ—R′ where X═Cl, Br; R=alkyl; R′=alkyl; n=1-20; m=1-20;
organosilanes of the type (RO)₃Si(CₙH₂ₙ₊₁), R′ₓ (RO)_{y}Si(CₙH₂ₙ₊₁), (RO)₃Si(CH₂)ₘ—R′, (R″)u(RO)vSi(C₂)ₘ—R′ where R=alkyl; R′=alkyl; n=1−20; m=1−20; x+y=3; x=1, 2; y=1, 2; u+v=2; u=1, 2; v=1, 2;
silazanes of the type R′R₂Si—NH—SiR₂R′ where R=alkyl, R′=alkyl, vinyl;
polysiloxanes of the type

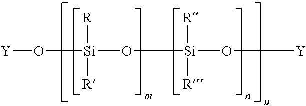

where R=alkyl, H; R′=alkyl, H; R″=alkyl, H; R′″=alkyl, H; Y═CH₃, H, CₚH₂ₚ₊₁ where p=1-20; Y=Si(CH₃)₃, Si(CH₃)₂H, Si(CH₃)₂OH, Si(CH₃)₂(OCH₃), Si(CH₃)₂(CₚH₂ₚ₊₁) where p=1−20; m=0, 1, 2, 3, . . . ∞; n=0, 1, 2, 3, . . . ∞; u=0, 1, 2, 3, . . . ∞; or
cyclic polysiloxanes of the type D3, D4 and/or D5.

The use of trimethoxyoctylsilane [(CH₃O)—Si—C₈H17], for example DYNASYLAN® OCTMO, Degussa AG, hexamethyl-disilazane, for example DYNASYLAN® HMDS, Degussa AG or polydimethylsiloxane as silanizing agent may be particularly preferred.

A further subject matter is a dispersion which comprises the hydrophobic zinc oxide particles according to the invention.

The liquid phase of the dispersion can be water, one or more organic solvents or an aqueous/organic combination, where the phases are miscible.

Liquid, organic phases may be, in particular, methanol, ethanol, n-propanol and isopropanol, butanol, octanol, cyclohexanol, acetone, butanone, cyclohexanone, ethyl acetate, glycol ester, diethyl ether, dibutyl ether, anisole, dioxane, tetrahydrofuran, mono-, di-, tri- and polyglycol ether, ethylene glycol, diethylene glycol, propylene glycol, dimethylacetamide, dimethylformamide, pyridine, N-methylpyrrolidine, acetonitrile, sulpholane, dimethyl sulphoxide, nitrobenzene, dichloromethane, chloroform, tetrachloromethane, ethylene chloride, pentane, hexane, heptane and octane, cyclohexane, benzines, petroleum ether, methylcyclohexane, decalin, benzene, toluene and xylenes. Ethanol, n- and isopropanol, ethylene glycol, hexane, heptane, toluene and o-, m- and p-xylene are particularly preferred as organic, liquid phase.

The dispersion according to the invention can further comprise pH regulators, surface-active additives and/or preservatives.

The content of hydrophobic zinc oxide particles according to the invention can preferably be 0.5 to 60% by weight. Particular preference is given to a dispersion comprising 10 to 50% by weight, in particular 35 to 45% by weight, of the hydrophobic zinc oxide particles according to the invention.

The average particle size in the dispersion can be varied within a wide range using appropriate dispersion units. These may, for example, be rotor-stator machines, high-energy mills, in which the particles grind themselves through collision with one another, planetary kneaders, stirred ball mills, ball mills operating as shaking unit, shaking panels, ultrasound units or combinations of the abovementioned units.

A particularly small particle size can be obtained by using rotor-stator machines and high-energy mills. The average particle size d₅₀ can here assume values of less than 180 nm, in particular less than 140 nm, determined by means of dynamic light scattering.

The invention further provides a sun protection formulation which comprises the hydrophobic zinc oxide particles according to the invention or the dispersion according to the invention.

These are present in the sun protection formulation usually in an amount of from 0.5 to 20% by weight, preferably 1 to 10% by weight and particularly preferably 3 to 8% by weight.

The sun protection formulation according to the invention can also comprise all water-soluble or oil-soluble UVA and UV-B filters known to the person skilled in the art.

For example
paraminobenzoic acid (PABA) and derivatives thereof, such as dimethyl-, ethyldihydroxypropyl-, ethylhexyldimethyl-, ethyl-, glyceryl- and 4-bis(polyethoxy)-PABA.
cinnamic acid esters, such as methyl cinnamate and methoxycinnamic acid esters, comprising octyl methoxycinnamate, ethyl methoxycinnamate, 2-ethylhexyl p-methoxycinnamate, isoamyl p-methoxycinnamate, diisopropyl cinnamate, 2-ethoxyethyl 4-methoxycinnamate, DEA methoxycinnamate (diethanolamine salt of p-methoxyhydroxycinnamic acid ester), diisopropyl methyl cinnamate;
benzophenones, such as 2,4-dihydroxy-, 2-hydroxy-4-methoxy-, 2,2′-dihydroxy-4,4′-dimethoxy-, 2,2′-dihydroxy-4-methoxy-, 2,2′,4,4′-tetrahydroxy-, 2-hydroxy-4-methoxy-4′-methylbenzophenones, sodium 2,2′-dihydroxy-4,4′-dimethoxy-5-sulphobenzophenones.
dibenzoylmethanes, such as butylmethoxydibenzoylmethane, in particular 4-tert-butyl-4′-methoxydibenzoylmethane;
2-phenylbenzimidazole-5-sulphonic acid and phenyldibenzimidazolesulphonic acid esters and salts thereof;
diphenylacrylates, such as alkyl alpha-cyano-beta,beta-diphenylacrylates, such as octocrylene;
triazines, such as 2,4,6-trianiline(p-carbo-2-ethylhexyl-1-oxy)-1,3,5-triazine, ethylhexyl-triazone and diethylhexylbutamidotriazone.
camphor derivatives, such as 4-methylbenzylidene- and 3-benzylidenecamphor and terephthalylidene-dicamphorsulphonic acid, benzylidenecamphorsulphonic acid, camphorbenzalkonium methosulphate and polyacrylamidomethylbenzylidenecamphor;
salicylates, such as dipropylene glycol, ethylene glycol, ethylhexyl, isopropylbenzyl, methyl, phenyl, 3,3,5-trimethyl and TEA salicylates (compound of 2-hydroxybenzoic acid and 2,2′,2″-nitrilotrisethanol);
esters of 2-aminobenzoic acid.

The sun protection formulation can further comprise compounds known to the person skilled in the art, such as organic solvents, thickeners, emulsifiers, softeners, antifoams, antioxidants, plant extracts, moisturizing agents, perfumes, preservatives and/or dyes, complexing agents, anionic, cationic, nonionic or amphoteric polymers or mixtures thereof, propellant gases and finely divided powders, including metal oxide pigments with a particle size of from 100 nm to 20 μm.

Suitable softeners are, in particular, avocado oil, cottonseed oil, behenyl alcohol, butyl myristate, butyl stearate, cetyl alcohol, cetyl palmitate, decyl oleate, di-n-butyl sebacate, thistle oil, eicosanyl alcohol, glyceryl monoricinoleate, hexyl laurate, isobutyl palmitate, isocetyl alcohol, isocetyl stearate, isopropyl isostearate, isopropyl laurate, isopropyl linoleate, isopropyl myristate, isopropyl palmitate, isopropyl stearate, isostearic acid, cocoa butter, coconut oil, lanolin, lauryl lactate, corn oil, myristyl lactate, myristyl myristate, evening primrose oil, octadecan-2-ol, olive oil, palmitic acid, palm kernel oil, polyethylene glycol, rapeseed oil, castor oil, sesame oil, soya oil, sunflower oil, stearic acid, stearyl alcohol, triethylene glycol.

Suitable emulsifiers are, in particular, glycerol monolaurate, glycerol monooleate, glycerol monostearate, PEG 1000 dilaurate, PEG 1500 dioleate, PEG 200 dilaurate, PEG 200 monostearate, PEG 300 monooleate, PEG 400 dioleate, PEG 400 monooleate, PEG 400 monostearate, PEG 4000 monostearate, PEG 600 monooleate, polyoxyethylene(4) sorbitol monostearate, polyoxyethylene(10) cetyl ether, polyoxyethylene(10)

monooleate,
polyoxyethylene(10) stearyl ether,
polyoxyethylene(12) lauryl ether,
polyoxyethylene(14) laurate,
polyoxyethylene(2) stearyl ether,
polyoxyethylene(20) cetyl ether,
polyoxyethylene(20) sorbitol monolaurate,
polyoxyethylene(20) sorbitol monooleate,
polyoxyethylene(20) sorbitol monopalmitate,
polyoxyethylene(20) sorbitol monostearate,
polyoxyethylene(20) sorbitol trioleate,
polyoxyethylene(20) sorbitol tristearate,
polyoxyethylene(20) stearyl ether,
polyoxyethylene(23) lauryl ether,
polyoxyethylene(25) oxypropylene monostearate,
polyoxyethylene(3.5) nonylphenol,
polyoxyethylene(4) lauryl ether,
polyoxyethylene(4) sorbitol monolaurate,
polyoxyethylene(5) monostearate,
polyoxyethylene(5) sorbitol monooleate,
polyoxyethylene(50) monostearate,
polyoxyethylene(8) monostearate,
polyoxyethylene(9.3) octylphenol, polyoxyethylene sorbitol lanolin derivatives, sorbitol monolaurate, sorbitol monooleate, sorbitol monopalmitate, sorbitol monostearate, sorbitol sesquioleate, sorbitol tristearate, sorbitol trioleate.

Suitable propellant gases may be propane, butane, isobutane, dimethyl ether and/or carbon dioxide.

Suitable finely divided powders may be chalk, talc, kaolin, colloidal silicon dioxide, sodium polyacrylate, tetraalkyl- and/or trialkylarylammonium smectites, magnesium aluminium silicates, montmorillonite, aluminium silicates, fumed silicon dioxide, and fumed titanium dioxide.

Typically, the sun protection composition according to the invention can be in the form of an emulsion (O/W, W/O or multiple), aqueous or aqueous-alcoholic gel or oil gel, and be supplied in the form of lotions, creams, milk sprays, mousse, stick or in other customary forms.

### EXAMPLES

### Analytical Methods

The BET surface area is determined in accordance with DIN 66131.

The transmission electron micrographs are obtained using a Hitachi TEM instrument, model H-75000-2. Using the CCD camera of the TEM instrument and subsequent image analysis, about 1000 to 2000 aggregates are evaluated. The definition of the parameters is in accordance with ASTM 3849-89. The shape analysis of the aggregates as circular, ellipsoidal, linear and branched is carried out in accordance with Herd et al., Rubber, Chem. Technol. 66 (1993) 491.

### Feed Materials:

Zinc oxide powder: the zinc oxide powders 1A-3A used are prepared by means of the process disclosed in DE-A-10343728. The analytical values of these powders are given in Table 1.

### Preparation of Hydrophobic Zinc Oxide Powders According to the Invention

The zinc oxide powder 1A is initially introduced into a mixer. With intense mixing, it is firstly optionally sprayed with water and then sprayed with a silanizing agent. When the spraying operation is complete, the mixture is afterstirred for about a further 15 minutes and then heat-treated.

The zinc oxides 2A and 3A are converted analogously. Feed materials and reaction conditions are given in Table 2.

### Sun Protection Formulations

The sun protection formulations according to the invention which, in the combination of hydrophobic ZnO according to the invention from Example 1B, have shown a synergistic effect with either OC=octocrylene, OMC=ethyl hexyl methoxycinnamate, ISA=phenylbenzimidazolesulphonic acid or BEMT=bisethylhexyloxy-methoxyphenyltriazine, are listed below. For statistical reasons, it is assumed that the SPF should be greater than or equal to two units higher than the sum SPFs of the individual formulations if the term synergism is used.

The SPF (sun protection factor) measurements are carried out in vitro using an optometrics SPF 290-S instrument.

### Examples 4A-C

In these examples, the standard formulation for W/O emulsions is used. The zinc oxide from Example 1B is introduced into the oil phase of the system. The additional fraction of the zinc oxide is subtracted from the oil phase (ethylhexyl stearate and mineral oil).
A standard formulation W/O emulsion with ZnO
B standard formulation W/O emulsion with OC
C standard formulation W/O emulsion with ZnO and OC

### Examples 5A-D

In these examples, the standard formulation for O/W emulsions is used. The zinc oxide from Example 1B is introduced into the oil phase of the system. The additional fraction of the zinc oxide is subtracted from the water phase (aqua). As an experiment, isostearic acid is used as surface modifier and pH stabilizer.
A standard formulation O/W emulsion with ZnO
B standard formulation O/W emulsion with OC
C standard formulation O/W emulsion with ZnO
D standard formulation O/W emulsion with ZnO+isostearic acid

### Examples 6A-C

In these examples, the standard formulation for W/O emulsions is used. The zinc oxide from Example 1B is introduced into the oil phase of the system. The additional fraction of the zinc oxide is subtracted from the oil phase (ethylhexyl stearate and mineral oil).
A standard formulation W/O emulsion with ZnO
B standard formulation W/O emulsion with OMC
C standard formulation W/O emulsion with ZnO and OMCA

### Examples 7A-D

In these examples, the standard formulation for O/W emulsions is used. The zinc oxide from Example 1B is introduced into the oil phase of the system. The additional fraction of the zinc oxide is subtracted from the water phase (aqua). As an experiment, isostearic acid is used as surface modifier and pH stabilizer.
A standard formulation O/W emulsion with ZnO
B standard formulation O/W emulsion with OMC
C standard formulation O/W emulsion with ZnO and OMC
D standard formulation O/W emulsion with ZnO, OMC and isostearic acid

### Examples 8A-C

In these examples, the standard formulation for W/0 emulsions is used. The zinc oxide from Example 1B is introduced into the oil phase of the system. The additional fraction of the zinc oxide is subtracted from the oil phase (ethylhexyl stearate and mineral oil).
A standard formulation W/O emulsion with ZnO
B standard formulation W/O emulsion with PISA
C standard formulation W/O emulsion with ZnO and PISA

### Examples 9A-D

In these examples, the standard formulation for O/W emulsions is used. The zinc oxide from Example 1B is introduced into the oil phase of the system. The additional fraction of the zinc oxide is subtracted from the water phase (aqua). As an experiment, isostearic acid is used.
A standard formulation O/W emulsion with ZnO
B standard formulation O/W emulsion with PISA
C standard formulation O/W emulsion with ZnO and PISA
D standard formulation O/W emulsion with ZnO, PISA and isostearic acid

### Examples 10A-C

In these examples, the standard formulation for W/0 emulsions is used. The zinc oxide from Example 1B is introduced into the oil phase of the system. The additional fraction of the zinc oxide is subtracted from the oil phase (C12-15 alkyl benzoate).
A standard formulation W/0 emulsion with ZnO
B standard formulation W/0 emulsion with BEMT
C standard formulation W/0 emulsion with ZnO and BEMT

### Examples 11A-D

In these examples, the standard formulation for O/W emulsions is used. The zinc oxide from Example 1B is introduced into the oil phase of the system. The additional fraction of the zinc oxide is subtracted from the water phase (aqua). As an experiment, isostearic acid is used as surface modifier and pH stabilizer.
A standard formulation O/W emulsion with ZnO
B standard formulation O/W emulsion with BEMT
C standard formulation O/W emulsion with ZnO and BEMT
D standard formulation O/W emulsion with ZnO, BEMT and isostearic acid

TABLE 1
Zinc oxide powders used

| | | Example | | |
|---|---|---|---|---|
| | | 1A | 2A | 3A |
| BET surface area | m²/g | 27 | 32 | 75 |
| Aggregates | | | | |
| circular | % | 4 | 1 | 9 |
| ellipsoidal | | 37 | 30 | 44 |
| linear | | 36 | 29 | 26 |
| branched | | 23 | 40 | 21 |
| Average aggregate | nm² | 4941 | 5502 | 2470 |
| area | | | | |
| Equivalent circle | nm | 66 | 110 | 33 |
| diameter of aggregate | | | | |
| Average aggregate | nm | 445 | 520 | 222 |
| circumference | | | | |

TABLE 2
Feed materials and adjustments

| | | Example | | |
|---|---|---|---|---|
| | | 1B | 2B | 3B |
| Zinc oxide | | 1A | 2A | 3A |
| Silanizing | | Octyl- | Octyl- | poly- |
| agent | | trimethoxy- | trimethoxy- | dimethyl- |
| | | silane | silane | siloxane |
| Fraction^{a)} | % by wt. | 1.5 | 3 | 2 |
| Fraction of | % by wt. | 0 | 0.2 | 0 |
| H₂O^{a)} | | | | |
| Temperature | ° C. | 140 | 140 | 160 |
| Time period | h | 1.2 | 1.2 | 1.4 |

^{a)}based on 100 parts ZnO

TABLE 3
Hydrophobic zinc oxide powders according to
the invention

| | | Example | | |
|---|---|---|---|---|
| | | 1B | 2B | 3B |
| BET surface | m²/g | 27 | 31 | 73 |
| C content | % by wt. | 0.8 | 1.1 | 0.7 |
| Si-alkyl | | Si-octyl | Si-octyl | Si-methyl |
| Si-(alkyl)₂ | | Si-(octyl)₂ | Si-(octyl)₂ | Si-(methyl)₂ |
| Aggregates | | | | |
| circular | % | 1 | 2 | 1 |
| ellipsoidal | | 18 | 21 | 17 |
| linear | | 26 | 24 | 29 |
| branched | | 55 | 53 | 53 |
| Average | nm² | 32 635 | 31 251 | 20 345 |
| aggregate | | | | |
| area | | | | |
| Equivalent | nm | 167 | 152 | 140 |
| circle | | | | |
| diameter of | | | | |
| aggregate | | | | |
| Average | nm | 1420 | 1240 | 980 |
| aggregate | | | | |
| circumference | | | | |

TABLE 4
W/O formulations - Example 4 (in %)

| Phase | INCI | 4A | 4B | 4C |
|---|---|---|---|---|
| A | Cetyl PEG/PPG-10/1 | 2.5 | 2.5 | 2.5 |
| | Dimethicone | | | |
| | Ethylhexyl | 12.5 | 12.5 | 10.0 |
| | Stearate | | | |
| | Mineral Oil | 12.5 | 12.5 | 10.0 |
| | Isostearic Acid | 1.0 | 1.0 | 1.0 |
| | Hydrogenated | 0.5 | 0.5 | 0.5 |
| | Castor Oil | | | |
| | Microcrystalline | 1.0 | 1.0 | 1.0 |
| | Wax | | | |
| | Octocrylene | | 5.0 | 5.0 |
| | Zinc Oxide | 5.0 | | 5.0 |
| B | Sodium Chloride | 0.5 | 0.5 | 0.5 |
| | Aqua | 64.45 | 64.45 | 64.45 |
| | 2-Bromo-2- | 0.05 | 0.05 | 0.05 |
| | Nitropropane-1,3- | | | |
| | diol | | | |
| SPF | | 2 | 3 | 6 |

TABLE 5
O/W formulations - Example 5 (in %)

| Phase | INCI | 5A | 5B | 5C | 5D |
|---|---|---|---|---|---|
| A | Ceteareth-15, | 2.5 | 2.5 | 2.5 | 2.5 |
| | Glyceryl Stearate | | | | |
| | Glyceryl Stearate | 1.0 | 1.0 | 1.0 | 1.0 |
| | Stearyl Alcohol | 2.0 | 2.0 | 2.0 | 2.0 |
| | C12-C15 Alkyl | 14.5 | 9.5 | 9.5 | 8.5 |
| | Benzoate | | | | |
| | Octocrylene | | 5.0 | 5.0 | 5.0 |
| | Zinc Oxide (w, c,) | 10.0 | | 10.0 | 10.0 |
| | Isostearic Acid | | | | 1.0 |
| B | Glycerine | 3.0 | 3.0 | 3.0 | 3.0 |
| | Aqua | 66.5 | 76.5 | 66.5 | 66.5 |
| | Chloroacetamide | 0.1 | 0.1 | 0.1 | 0.1 |
| C | Xanthan Gum | 0.4 | 0.4 | 0.4 | 0.4 |
| SPF | | 2 | 3 | 8 | 9 |

TABLE 6
W/O formulations - Example 6 (in %)

| Phase | INCI | 6A | 6B | 6C |
|---|---|---|---|---|
| A | Cetyl PEG/PPG-10/1 | 2.5 | 2.5 | 2.5 |
| | Dimethicone | | | |
| | Ethylhexyl Stearate | 12.5 | 12.5 | 10.0 |
| | Mineral Oil | 12.5 | 15.5 | 10.0 |
| | Isostearic Acid | 1.0 | 1.0 | 1.0 |
| | Hydrogenated Castor | 0.5 | 0.5 | 0.5 |
| | Oil | | | |
| | Microcrystalline Wax | 1.0 | 1.0 | 1.0 |
| | Ethylhexyl | | 5.0 | 5.0 |
| | Methoxycinnamate | | | |
| | Zinc Oxide (w, c,) | 5.0 | | 5.0 |
| B | Sodium Chloride | 0.5 | 0.5 | 0.5 |
| | Aqua | 64.45 | 64.45 | 64.45 |
| | 2-Bromo-2- | 0.05 | 0.05 | 0.05 |
| | Nitropropane-1,3-diol | | | |
| SPF | | 2 | 7 | 13 |

TABLE 7
O/W formulations - Example 7 (in %)

| Phase | INCI | 7A | 7B | 7C | 7D |
|---|---|---|---|---|---|
| A | Ceteareth-15, | 2.5 | 2.5 | 2.5 | 2.5 |
| | Glyceryl Stearate | | | | |
| | Glyceryl Stearate | 1.0 | 1.0 | 1.0 | 1.0 |
| | Stearyl Alcohol | 2.0 | 2.0 | 2.0 | 2.0 |
| | C12-C15 Alkyl | 14.5 | 9.5 | 9.5 | 8.5 |
| | Benzoate | | | | |
| | Ethylhexyl | | 5.0 | 5.0 | 5.0 |
| | Methoxycinnamate | | | | |
| | Zinc Oxide (w, c,) | 10.0 | | 10.0 | 10.0 |
| | Isostearic Acid | | | | 1.0 |
| B | Glycerine | 3.0 | 3.0 | 3.0 | 3.0 |
| | Aqua | 66.5 | 76.5 | 66.5 | 66.5 |
| | Chloroacetamide | 0.1 | 0.1 | 0.1 | 0.1 |
| C | Xanthan Gum | 0.4 | 0.4 | 0.4 | 0.4 |
| SPF | | 2 | 6 | 11 | 16 |

TABLE 8
W/O formulations - Example 8 (in %)

| Phase | INCI | 8A | 8B | 8C |
|---|---|---|---|---|
| A | Cetyl PEG/PPG-10/1 | 2.5 | 2.5 | 2.5 |
| | Dimethicone | | | |
| | Ethylhexyl Stearate | 12.5 | 15.0 | 12.5 |
| | Mineral Oil | 12.5 | 15.0 | 12.5 |
| | Isostearic Acid | 1.0 | 1.0 | 1.0 |
| | Hydrogenated Castor Oil | 0.5 | 0.5 | 0.5 |
| | Microcrystalline Wax | 1.0 | 1.0 | 1.0 |
| | Zinc Oxide (w, c,) | 5.0 | | 5.0 |
| B | Sodium Chloride | 0.5 | 0.5 | 0.5 |
| | Aqua | 64.45 | 49.45 | 49.45 |
| | 2-Bromo-2-Nitropropane- | 0.05 | 0.05 | 0.05 |
| | 1,3-diol | | | |
| | Phenylbenzimidazole | | 15.0 | 15.0 |
| | Sulfonic Acid (20% Aqua) | | | |
| SPF | | 2 | 5 | 9 |

TABLE 9
O/W formulations - Example 9 (in %)

| Phase | INCI | 9A | 9B | 9C | 9D |
|---|---|---|---|---|---|
| A | Ceteareth-15, Glyceryl | 2.5 | 2.5 | 2.5 | 2.5 |
| | Stearate | | | | |
| | Glyceryl Stearate | 1.0 | 1.0 | 1.0 | 1.0 |
| | Stearyl Alcohol | 2.0 | 2.0 | 2.0 | 2.0 |
| | C12-C15 Alkyl Benzoate | 14.5 | 14.5 | 14.5 | 13.5 |
| | Zinc Oxide (w, c,) | 10.0 | | 10.0 | 10.0 |
| | Isostearic Acid | | | | 1.0 |
| B | Glycerine | 3.0 | 3.0 | 3.0 | 3.0 |
| | Aqua | 66.5 | 61.5 | 51.5 | 51.5 |
| | Chloroacetamide | 0.1 | 0.1 | 0.1 | 0.1 |
| | Phenylbenzimidazole | | 15.0 | 15.0 | 15.0 |
| | Sulfonic Acid (20% Aqua) | | | | |
| C | Xanthan Gum | 0.4 | 0.4 | 0.4 | 0.4 |
| SPF | | 2 | 5 | 11 | 15 |

TABLE 10
W/O formulations - Example 10 (in %)

| Phase | INCI | 10A | 10B | 10C |
|---|---|---|---|---|
| A | Cetyl PEG/PPG-10/1 | 2.5 | 2.5 | 2.5 |
| | Dimethicone | | | |
| | C12-15 Alkyl Benzoate | 27.0 | 25.0 | 22.0 |
| | Isostearic Acid | 1.0 | 1.0 | 1.0 |
| | Hydrogenated Castor Oil | 0.5 | 0.5 | 0.5 |
| | Microcrystalline Wax | 1.0 | 1.0 | 1.0 |
| | Bis-Ethylhexyloxyphenol | 3.0 | | 3.0 |
| | Methoxyphenyl Triazine | | | |
| | Zinc Oxide (w, c,) | | 5.0 | 5.0 |
| B | Sodium Chloride | 0.5 | 0.5 | 0.5 |
| | Aqua | 64.45 | 64.45 | 64.45 |
| | 2-Bromo-2-Nitropropane- | 0.05 | 0.05 | 0.05 |
| | 1,3-diol | | | |
| SPF | | 2 | 8 | 13 |

TABLE 11
O/W formulations - Example 11 (in %)

| Phase | INCI | 11A | 11B | 11C | 11D |
|---|---|---|---|---|---|
| A | Ceteareth-15, Glyceryl | 2.5 | 2.5 | 2.5 | 2.5 |
| | Stearate | | | | |
| | Glyceryl Stearate | 1.0 | 1.0 | 1.0 | 1.0 |
| | Stearyl Alcohol | 2.0 | 2.0 | 2.0 | 2.0 |
| | C12-C15 Alkyl Benzoate | 14.5 | 12.5 | 12.5 | 11.5 |
| | Bis-Ethylhexyloxyphenol | | 2.0 | 2.0 | 2.0 |
| | Methoxyphenyl Triazine | | | | |
| | Zinc Oxide (w, c,) | 10.0 | | 10.0 | 10.0 |
| | Isostearic Acid | | | | 1.0 |
| B | Glycerine | 3.0 | 3.0 | 3.0 | 3.0 |
| | Aqua | 66.5 | 76.5 | 66.5 | 66.5 |
| | Chloroacetamide | 0.1 | 0.1 | 0.1 | 0.1 |
| C | Xanthan Gum | 0.4 | 0.4 | 0.4 | 0.4 |
| SPF | | 2 | 3 | 6 | 8 |

1. A hydrophobic zinc oxide powder in the form of partially or completely coated aggregates of zinc oxide primary particles with a carbon content ranging from 0.4 to 1.5% by weight, based on the hydrophobic zinc oxide powder, and a BET surface area ranging from 25 to 100 m²/g, where
the coated aggregates are present in an amount of 0 to <10% in a circular form, 15 to <30% in an ellipsoidal form, 15 to <30% in a linear form, 40 to <70% in a branched form and the sum of the different forms is 100% and
the coating comprises chemically bonded linear and/or branched alkylsilyl groups having 1 to 20 carbon atoms.

2. The hydrophobic zinc oxide powder according to claim 1, wherein the aggregates have an average projected aggregate area of 8000-30 000 nm², an equivalent circle diameter (ECD) of 70-300 nm and an average circumference of 500-2000 nm.

3. The hydrophobic zinc oxide powder according to claim 1, wherein the proportion of lead is at most 20 ppm, of arsenic at most 3 ppm, of cadmium at most 15 ppm, of iron at most 200 ppm, of antimony at most 1 ppm and of mercury at most 1 ppm.

4. The hydrophobic zinc oxide powder according to claim 1, wherein
the alkylsilyl groups chemically bonded to the aggregate surfaces contain 1 to 8 carbon atoms,
the aggregates are present in the circular form in an amount of 0 to 5%, the ellipsoidal form of 20 to 25%, the linear form of 20 to 25% and the branched form of 45 to 60%, where the sum of the different forms is 100%,
the carbon content ranges from 0.6 to 1.2% by weight, based on the hydrophobic zinc oxide powder, and
the BET surface area is 25 to 40 m²/g.

5. A process for the preparation of the hydrophobic zinc oxide powder according to claim 1, wherein a zinc oxide powder in the form of aggregated primary particles having a BET surface area of 25 and 100 m²/g, in which the aggregates are present in an amount of 0-10% in a circular form, 30-50% in an ellipsoidal form, 30-50% in a linear form and 20-30% in a branched form is sprayed with one or more silanizing agents which contain 1 to 20 carbon atoms, which optionally are dissolved in an organic solvent, and the mixture is then thermally treated at a temperature of from 120 to 200° C. over a period ranging from 0.5 to 2 hours.

6. The process according to claim 5, wherein the silanizing agent is selected from the group consisting of
haloorganosilanes of the type X₃Si(CₙH₂ₙ₊₁), X₂(R′)Si(CₙH₂ₙ₊₁), X(R′)₂Si(CₙH₂ₙ₊₁), X₃Si(CH₂)ₘ—R′(R)X₂Si(CH₂)ₘ—R′, (R)₂XSi(CH₂)ₘ—R′ where X═Cl, Br; R=alkyl; R′=alkyl; n=1−20; m=1−20;
the organosilanes of the type (RO)₃Si(CₙH₂ₙ₊₁), R′ₓ(RO)_{y}Si(CₙH₂ₙ₊₁), (RO)₃Si(CH₂)ₘ—R′, (R″)ᵤ(RO)ᵥSi(CH₂)ₘ—R′ where R=alkyl; R′=alkyl; n=1−20; m=1−20; x+y=3; x=1, 2; y=1, 2; u+v=2; u=1, 2; v=1, 2;
silazanes of the type R′R₂Si—NH—SiR₂R′ where R=alkyl, R′=alkyl, vinyl;
polysiloxanes of the type 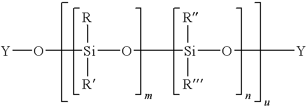
where R=alkyl, H; R′=alkyl, H; R″=alkyl, H; R′″=alkyl, H; Y═CH₃, H, CₚH₂ₚ₊₁) where p=1−20; Y═Si(CH₃)₃, Si(CH₃)₂H, Si(CH₃)₂OH, Si(CH₃)₂(OCH₃), Si(CH₃)₂(CₚH₂ₚ₊₁) where p=1−20; m=0, 1, 2, 3, . . . ∞; n=0, 1, 2, 3, . . . u=0, 1, 2, 3, . . . ∞; and
cyclic polysiloxanes of the type D3, D4 and/or D5.

7. A dispersion comprising the hydrophobic zinc oxide powder according to claim 1.

8. A sun protection formulation comprising the hydrophobic zinc oxide particles according to claims 1.

9. A sun protection formulation comprising the hydrophobic zinc oxide particle dispersion according to claim 7.
